(19) United States
(12) Patent Application Publication
(10) Pub. No.: US 2021/0350940 A1
(43) Pub. Date: Nov. 11, 2021

(54) REVERSIBLE SHARING OF ELECTRONIC MEDICAL DATA USING DATABASES

(71) Applicant: International Business Machines Corporation, Armonk, NY (US)

(72) Inventors: Patrick Arthur Fagan, Naas (IE); Brian Lavin, Dublin (IE); ELAINE HUGHES, Blarney (IE); Karie Lynn Kelly, Carrollton, TX (US); LUKE SCOLLAN, Bettystown (IE)

(21) Appl. No.: 16/871,547

(22) Filed: May 11, 2020

(51) Int. Cl. G16H 80/00; G16H 10/60; G16H 50/30

(52) U.S. Cl. CPC G16H 80/00; G16H 50/30; G16H 10/60

(57) ABSTRACT

Techniques are provided for managing sharing of data for users. An authorization is received from a user to share user data between a plurality of organizations. The user data is stored in one or more isolated databases. A medical records data management system generates a shared database comprising content including the user data to provide access to the user data to the plurality of organizations. The shared database comprises a data structure for associating the user data an origin of the user data. When revocation by a user to withdraw authorization to share data between the plurality of organizations is received, the content of the shared database is resolved into the one or more isolated databases, and the content is assigned to the isolated database based upon the origin of the user data in the data structure. Access is closed to the shared database.

### 1. TECHNICAL FIELD

Present invention embodiments relate to management of electronic medical data, and in particular, to reversible sharing of electronic medical data using databases.

### 2. DISCUSSION OF THE RELATED ART

In recent years, organizations have migrated to software-based platforms for the management of medical and other data. In the medical sector, patients may seek assistance from different physicians associated with different medical organizations, which leads to patient data distributed across a variety of isolated sources. Patients may complete legal forms to provide consent for sharing personal information in order to allow physicians or other personnel to access their medical data at other medical organizations.

However, patients may later revoke consent, and information may no longer be shared. Traditional approaches of dissolving shared information involve deleting or removing all shared files, which leads to loss of information.

### SUMMARY

According to embodiments of the present invention, methods, systems, and computer readable media are provided for managing access to medical information. An authorization is received from a user to share user data between a plurality of organizations, wherein the user data is stored in one or more isolated databases. A data management system generates a shared database comprising content including the user data to provide access to the user data to the plurality of organizations, wherein the shared database comprises a data structure for associating the user data with an origin of the user data. A revocation is received from a user that withdraws authorization to share data between the plurality of organizations. The content of the shared database is resolved into the one or more isolated databases based upon the origin of the user data in the data structure. Access is closed to the shared database.

It is to be understood that the Summary is not intended to identify key or essential features of embodiments of the present disclosure, nor is it intended to be used to limit the scope of the present disclosure. Other features of the present disclosure will become easily comprehensible through the description below.

### BRIEF DESCRIPTION OF THE DRAWINGS

Generally, like reference numerals in the various figures are utilized to designate like components.

FIG. 1A is a diagrammatic illustration of an example computing environment for a data management system, according to an embodiment of the present invention.

FIG. 1B is an example computing device for the computing environment of FIG. 1A, according to an embodiment of the present invention.

FIG. 2 is an illustration showing isolated user data for a patient and for shared user data for a patient, according to an embodiment of the present invention.

FIG. 3 is an illustration of data flow for sharing user data, according to an embodiment of the present invention.

FIG. 4 shows a flowchart for generating shared user data, according to an embodiment of the present invention.

FIG. 5 is an illustration of data flow for resolving shared user data into data subsets based on origin, according to an embodiment of the present invention.

FIG. 6 shows a flowchart for resolving shared user data into subsets, according to an embodiment of the present invention.

FIGS. 7A and 7B show example implementations of shared databases, according to aspects of the present invention.

FIG. 8 shows a flowchart for managing shared user data, according to an embodiment of the present invention.

### DETAILED DESCRIPTION

According to the aspects disclosed herein, methods, systems, and computer readable media for reversibly sharing user data are provided. These techniques generate shared data and resolve shared data into data subsets based on the origin of the shared data in order to provide data subsets to their respective organizational owners.

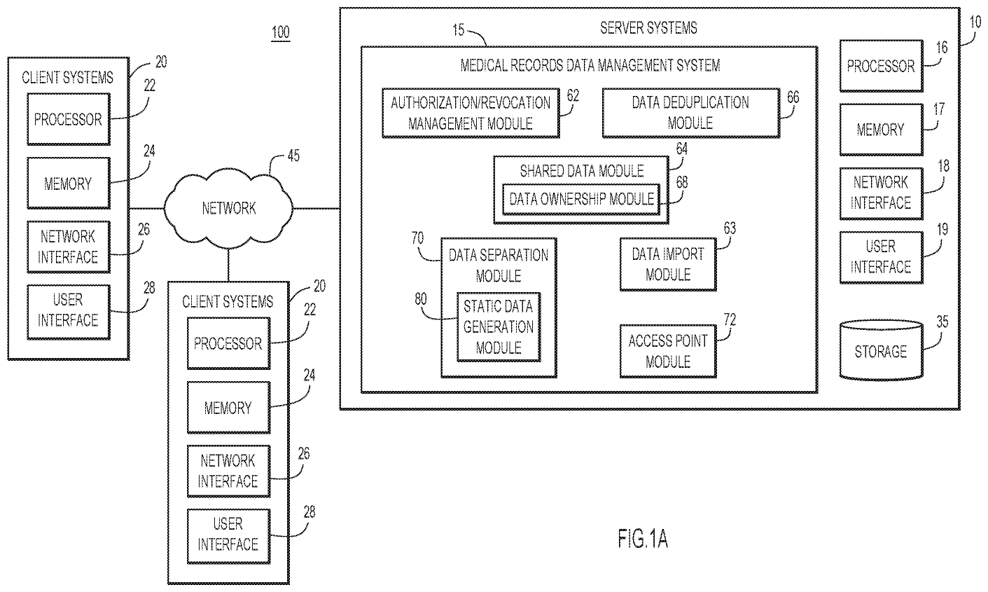

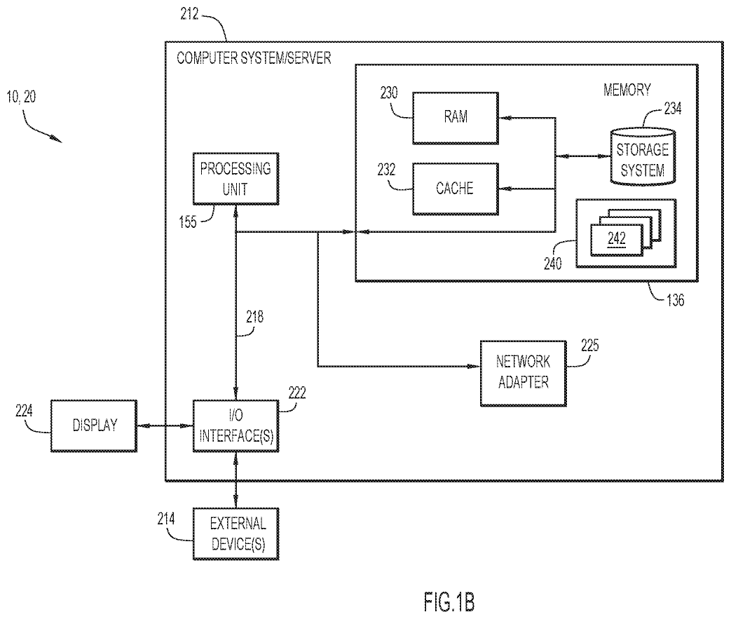

An example environment for use with present invention embodiments is illustrated in FIG. 1A. Specifically, environment 100 includes one or more server systems 10, one or more client or end-user systems 20 and a network 45. Server systems 10 and client systems 20 may be remote from each other and may communicate over network 45. The network may be implemented by any number of any suitable communications media, such as a wide area network (WAN), a local area network (LAN), Internet, Intranet, etc. Alternatively, server systems 10 and client systems 20, may be local to each other, and may communicate via any appropriate local communication medium, such as local area network (LAN), hardwire, wireless link, Intranet, etc.

Client systems 20 enable users to provide user data (e.g., medical records, etc.) to server systems 10 and to obtain user data from server systems 10.

Server systems 10 may comprise a storage database 35 that stores various types of information (e.g., user data including medical records, origin of medical records, authorizations, revocations, etc.) for the analysis. Storage 35 may include any suitable information in a structured, semi-structured, or unstructured format. For large volumes of medical data, platforms geared towards management of large data sets across clusters of computers along with structured query language interfaces (e.g., BigSQL, etc.) and/or applications for generating logically linked databases may be utilized.

Storage database 35 may be implemented by any conventional or other database or storage unit, may be local to or remote from server systems 10 and client systems 20 and may communicate via any appropriate communication medium, such as local area network (LAN), wide area network (WAN), Internet, hardwire, wireless link, Intranet, etc. The client systems may present a graphical user interface, such as a GUI, etc., or other interface, such as command line prompts, menu screens, etc., to solicit user data and to return resolved user data to their organizational owners.

Server systems 10 and client systems 20 may be implemented by any conventional or other computer systems preferably equipped with a display or monitor, a base (including at least one hardware processor (e.g., microprocessor, controller, central processing unit (CPU), etc.), one or more memories and/or internal or external network interfaces or communications devices (e.g., modem, network cards, etc.), optional input devices (e.g., a keyboard, mouse or other input device), and any commercially available and custom software (e.g., server/communications software, medical records data management system, browser/interface software, etc.). By way of example, the server/client includes at least one processor 16, 22, one or more memories 17, 24 and/or internal or external network interfaces or communications devices 18, 26 such as a modem or network cards, and a user interface 19, 28 etc. The optional input devices may include a keyboard, mouse, or other input device. The client system may be any suitable device, including but not limited to desktops, laptops, tablets, etc. or any other device capable of serving as an interface to a data management system.

Alternatively, one or more client systems 20 may perform the operations of servers systems 10 in a stand-alone mode of operation. For example, the client system may store or have access to medical records data management system 15. The graphical user or other interfaces 19, 28, such as a GUI, command line prompts, menu screens, etc., solicits user data from organizations, and provides access to shared user data for organizations authorized to view shared information.

Medical records data management system 15 may include one or more modules or units to perform the various functions of present invention embodiments described herein. The various modules (e.g., medical records data management system 15, comprising authorization/revocation management module 62, data import module 63, shared data module 64, data deduplication module 66, data separation module 70, access point module 72, etc.), may be implemented by any combination of any quantity of software and/or hardware modules or units, and may reside within memory 17 of the server for execution by processor 16. These modules are described in additional detail below.

Authorization/revocation management module 62 tracks authorizations provided by a user (e.g., patient) to share user data (e.g., medical data) as well as revocations to withdraw authorization to share user data. In some aspects, a patient may provide authorization to share all available user data or a portion of available user data. Similarly, a patient may revoke sharing user data for all shared medical data or a portion of shared user data (e.g., based on origin or content of the medical data).

Data import module 63 imports user data from other organizations if information to be shared is not accessible to medical records data management system 15. In some aspects, a local copy of user data may be stored in storage 35. For example, imported user data may be stored as an isolated database for each organization sharing data.

Shared data module 64 generates a database comprising shared user data. In some aspects, the database may be a logically linked database. For example, imported medical records may be stored locally as isolated databases. To generate the shared database, shared data module 64 may package a plurality of isolated databases comprising medical records from an organization in a wrapper, effectively logically linking each database within the wrapper to provide visibility into all linked databases. Logically linked databases combine the contents of specified database tables or records, and may be used with any number of executable programs. Within a database, tables or records may be linked to each other using foreign key relationships, and data may be read from database tables that are part of these linked structures. To discontinue sharing, links between databases may be disconnected.

Thus, logically linked databases may be generated by placing a file wrapper around isolated databases. Applications may access the underlying data through the wrapper. Contents of the individual databases may be linked by linking database objects. In some aspects, the file wrapper may track the origin, via a data structure, of the user data. Each logically linked database may be mapped to or associated with one of the organizations. Additionally, database may refer to a fragment of a database rather than a database in its entirety.

In aspects, by providing a wrapper around copies of data, users can edit data (e.g., goals, shared notes, etc.) while having a single shared instance (e.g., to record care plan). The wrapper can visualize shared components into systems in order to identify which data is added by an organization.

In other aspects, a shared database may import each isolated database into a combined database. The shared database may comprise a data structure to identify the origin of the shared data (e.g., the organization, physician, database from which the data was obtained). For user data added to the shared user data, the data structure will track the origin of the added data. The data structure allows the user data (on a per record or per field basis) to be resolved should authorization be revoked. In this embodiment, data from isolated databases may be read into a combined database, rather than linked, and may be read out to regenerate isolated databases.

Data deduplication module 66 may deduplicate certain types of conserved information (e.g., demographics, health data, etc.). For example, data pertaining to patient demographics and to prior medical studies (e.g., test results, etc.) are conserved and should be consistent across provider platforms. Information that is not conserved is not deduplicated (e.g., care plan data, including goals, notes, assessments, etc.) as this data may vary among different providers).

Data ownership module 68, which may comprise the data structure to resolve origin of user data, associates user data with its respective origin (e.g., organization, physician, entity that created the medical record, database from which the data was obtained, etc.). For user data that is modified in the shared environment or newly added, data ownership module 68 associates modified or added data with its respective origin of information (e.g., the organization, physician, or database from which the data was obtained, via the data structure).

Data separation module 70 separates shared user data for return to its origin. In some aspects, data separation module 70 may include a static data generation module 80, which creates a copy of the shared data at the time authorization is revoked. This data can no longer be modified, and acts as an archival copy of the shared data when authorization is revoked. The static copy may be provided to all organizations contributing shared data. Portions of shared data that are returned to their respective owners/originators may be further modified.

Access point module 72 provides access to the medical records. When the records are not shared, access point module routes access to the relevant isolated database. When user data is shared, access point module 72 routes access directly to the shared database.

Client systems 20 and server systems 10 may be implemented by any suitable computing device, such as computing device 212 shown in FIG. 1B for computing environment 100. This example is not intended to suggest any limitation as to the scope of use or functionality of embodiments of the invention described herein. Regardless, computing device 212 is capable of being implemented and/or performing any of the functionality set forth herein.

In the computing device, there is a computer system which is operational with numerous other general purpose or special purpose computing system environments or configurations. Examples of computing systems, environments, and/or configurations that may be suitable for use with the computer system include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 212 may be described in the general context of computer system executable instructions, such as program modules (e.g., medical records data management system 15 and its corresponding modules), being executed by a computer system. Generally, program modules may include routines, programs, objects, components, logic, data structures, and so on that perform particular tasks or implement particular abstract data types.

Computer system 212 is shown in the form of a general-purpose computing device. The components of computer system 212 may include, but are not limited to, one or more processors or processing units 155, a system memory 136, and a bus 218 that couples various system components including system memory 136 to processor 155.

Bus 218 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 212 typically includes a variety of computer system readable media. Such media may be any available media that is accessible by computer system 212, and it includes both volatile and non-volatile media, removable and non-removable media.

System memory 136 can include computer system readable media in the form of volatile memory, such as random access memory (RAM) 230 and/or cache memory 232. Computer system 212 may further include other removable/non-removable, volatile/non-volatile computer system storage media. By way of example only, storage system 234 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 218 by one or more data media interfaces. As will be further depicted and described below, memory 136 may include at least one program product having a set (e.g., at least one) of program modules that are configured to carry out the functions of embodiments of the invention.

Program/utility 240, having a set (at least one) of program modules 242 (e.g., medical records data management system 15 and corresponding modules, etc.) may be stored in memory 136 by way of example, and not limitation, as well as an operating system, one or more application programs, other program modules, and program data. Each of the operating system, one or more application programs, other program modules, and program data or some combination thereof, may include an implementation of a networking environment. Program modules 242 generally carry out the functions and/or methodologies of embodiments of the invention as described herein.

Computer system 212 may also communicate with one or more external devices 214 such as a keyboard, a pointing device, a display 224, etc.; one or more devices that enable a user to interact with computer system 212; and/or any devices (e.g., network card, modem, etc.) that enable computer system 212 to communicate with one or more other computing devices. Such communication can occur via Input/Output (I/O) interfaces 222. Still yet, computer system 212 can communicate with one or more networks such as a local area network (LAN), a general wide area network (WAN), and/or a public network (e.g., the Internet) via network adapter 225. As depicted, network adapter 225 communicates with the other components of computer system 212 via bus 218. It should be understood that although not shown, other hardware and/or software components could be used in conjunction with computer system 212. Examples, include, but are not limited to: microcode, device drivers, redundant processing units, external disk drive arrays, RAID systems, tape drives, and data archival storage systems, etc.

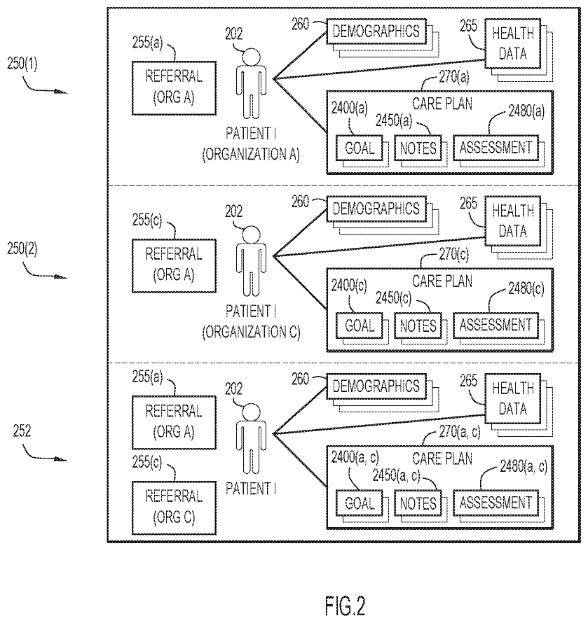

FIG. 2 is an illustration showing isolated user data 250(1) and 250(2) from different organizations that has not been shared, and shared user data 252 that has been shared.

Patient I 202 may receive a referral to organization A 255(a), and demographics 260 and health data 265 may be sent to a data management system housing data for organization A. In this instance, patient 202 is managed as an isolated patient associated with organization A.

The same patient I 202 may receive a referral to organization C 255(c), and demographic 260 and health data 265 may be sent to a data management system for organization C. In this instance, the patient is managed as an isolated patient associated with organization C.

Accordingly, patient I has isolated data at two different organizations (organization A and organization C), and user data between organizations C and A is not shared. Each organization has access to only the user data (e.g., patient data) that is available to that partner/provider. In this example, a single patient has multiple isolated instances of data (e.g., pertaining to health care plans), and such data is not visible outside the organization that created it.

In a shared data environment, user data from different organizations is shared between organizations. In this embodiment, patient I is referred to organization C. Demographic and health data is sent to the data management system from organization C. Patient I is also referred to organization A. Demographic and health data is sent to the data management system from organization A. If authorization is provided by the patient to share user data, then both organizations can access the shared user data. In this example, patient data from organization A would be available to providers of organization C and vice-versa as shown in shared user data 252.

For isolated user data 250(1) and 250(2) stored in the data management system, the system may create a logical database linking the user data. All or a portion of available patient data may be shared by one or more organizations.

In this example, demographics 260 and health data 265 are data that is specific to the patient and is uniform across all databases. In aspects, the medical records data management system 15 may verify that this type of data is consistent, and may flag any differences for review and resolution. Data from isolated patient care plans 270(a) and 270(c) may be merged and stored in shared database as care plans 270(a,c). This data may include goals 2400(a) and 2400(c) which correspond to goals 2400(a,c) in the shared database. Notes 2450(a) and 2450(c) correspond to notes 2400(a,c) in the shared database. Assessment 2480(a) and 2480(c) correspond to assessment 2480(a,c) in the shared database. Care plans from different organizations may contain different information, and therefore, this information is stored without deduplication.

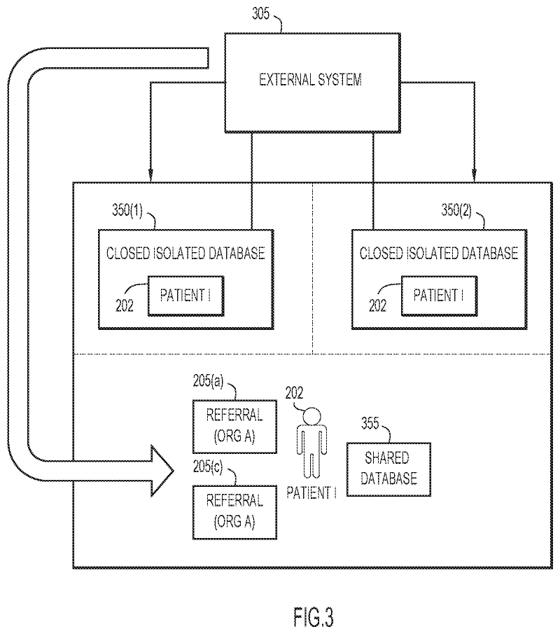

FIG. 3 is an illustration of generating a shared database that shares user data between two different organizations. In some aspects, external system 305 is a secure, cloud-based platform, housing longitudinal electronic health records data, with analytic tools to analyze healthcare data. The external system may transmit user data to the shared database 355 when operational. (Alternatively, the external system may transmit user data to isolated databases 350(1) and 350(2) when the shared database is closed.)

For the examples provided herein, an arrangement is shown in which data from the external system 305 (e.g., client device 10) is provided to the medical records data management system 15 (servers systems 10). In these examples, data is provided from the external system to the shared database 355 of the medical records data management system 15, when the shared database is present and the isolated databases are closed 350(1) and 350(2). However, these examples are not intended to be limiting, and also include embodiments in which the isolated databases may be present in client devices.

When a patient with one or more existing health care providers/plans, grants authorization for data sharing, a shared database is generated with consolidated demographic and health data. The medical records data management system will include user data from partner A and partner C at different organizations, to generate a consolidated database with shared user data (e.g., patient health care data).

In this example, external systems 305 provides localized copies of user data to the medical records data management system, which is stored as isolated databases. Once user data is shared, access to patient data (for a patient and their care plans associated with the individual partners) via the isolated databases 350(1) and 350(2) is closed, and the shared database 355 is generated. Additions to the shared data are made within the data management system, and added data may be tracked with regard to origin (e.g., the organization that adds the new information).

In general, database may refer to any form for storing structured information (e.g., tables, records, spreadsheets, structured databases, unstructured databases, etc.) or semi-structured information.

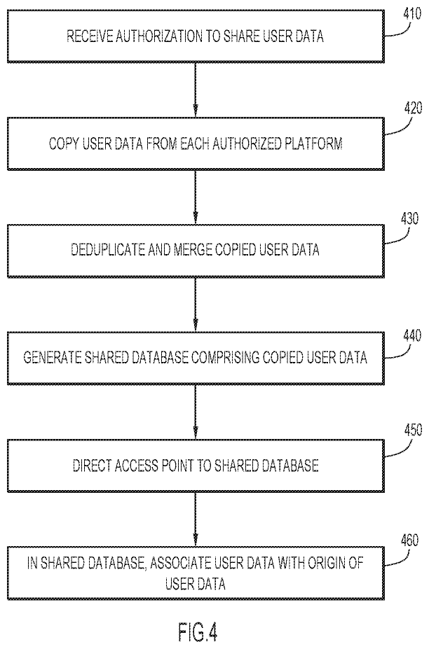

FIG. 4 is a flowchart of operations associated with sharing data from different organizations. At operation 410, authorization is received from the patient to share user data. At operation 420, if the information is stored in a separate system, the patient data may be copied locally from each authorized platform to the data management system, e.g., as an isolated database. Ownership of the user data is determined by the organization from which the data is copied.

At operation 430, portions of the data (e.g., demographics, health data) may be deduplicated. Deduplicated information includes data which is conserved across platforms. In some cases, conflicts may be identified by the system and resolved in an automated manner or based on user input. Other types of information (e.g., notes, goals, assessments) are not deduplicated.

At operation 440, a shared database (e.g., logical database) is generated, linking the user data in each respective database using appropriate identifiers (e.g., foreign keys, etc.). In aspects, data is retrieved by application programs that access data from tables or other structures provided in the shared database. Alternatively, user data from isolated databases may be read into a combined database, along with origin of data, for sharing.

At operation 450, the medical records data management system changes the access point from the isolated databases to the shared database, allowing access to the shared information.

At operation 460, additional user data may be added to the shared data. In this case, the system tracks the organization that contributed the shared data, and thereby, associates ownership of the additional information with that organization.

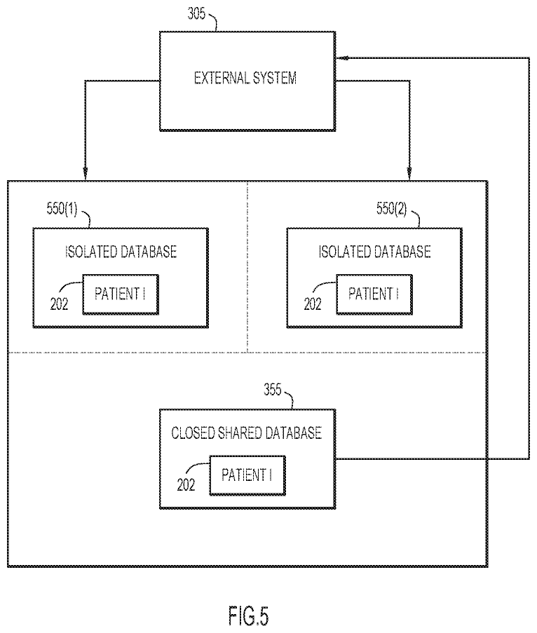

FIG. 5 shows an illustration of revocation of authorization to share data. In this example, a user revokes authorization for shared data, and the management system separates the data into its respective components to be returned to organizations that provided the data. In this example, two organizations contribute shared user data. After revocation, the shared data is separated and returned to external systems 305. Alternatively or additionally, the data may be stored as isolated databases 550(1) and 550(2), wherein each isolated database corresponds to an organization's data.

In this embodiment, the owner of the shared data is identified, and the data is returned to its respective owner. For example, a patient record generated by the medical records data management system (for each organization) with demographic and health data is returned to the organization owning the data. If this data has been modified for consistency, the data returned to the user may be corrected as compared to the original version. Information added as shared data in the shared database is also returned to its respective organizational owner (e.g., the organization that added the information). Ownership is determined based on the data structure in which origin information is stored for the components of the shared data.

The shared database 555 comprising the shared user data is ‘closed’ (e.g., access is no longer permitted). A snapshot of the user data is obtained at the time that the shared database is closed, and returned to all organizations (e.g., via external system 305 and/or via isolated databases 550(1) and 550(2)) as a static copy of the data that was shared, e.g., as reference data that cannot be modified or altered by organizations not owning the data. Additionally, modifiable data is returned to each organization that originally contributed the data. For example, if organization A contributed goal A, notes A and assessment A as well as added data A, then editable versions of this information are returned to their respective origin, in this case organization A. Thus, this process is the reverse of FIG. 3.

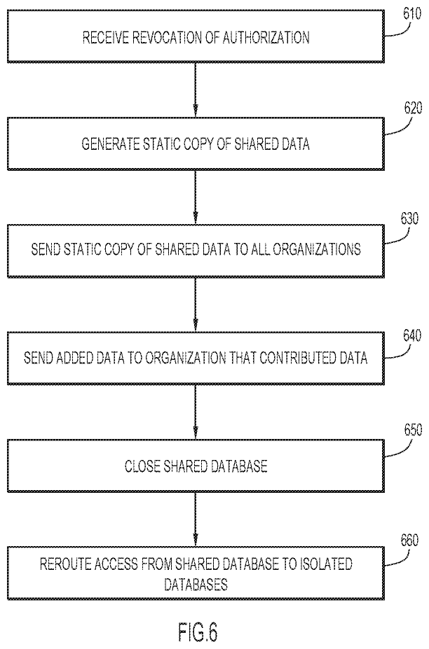

FIG. 6 is a flowchart showing operations involved with resolving data. At operation 610, revocation of authorization to share data is received. At operation 620, a static copy of the shared data is generated. At operation 630, the static copy is sent to each organization associated with the shared data. At operation 640, portions of data are returned to the organization that contributed the data. User data added after data sharing is activated may be similarly resolved. At operation 650, the shared database is closed. In aspects, logical links that form the logical database are disconnected and/or wrappers are removed to separate user data into isolated databases. Alternatively, user data from a combined database may be read into a plurality of isolated databases, based on origin of data. At operation 660, access is rerouted from the shared database to isolated databases.

Thus, when data sharing is implemented, user data is associated with its origin (e.g., an organization creating the data, etc.). When added user data is created, it is associated with the organization that generated the data. Accordingly, even though data is shared, every data element has an owner, and each sub-element of user data has an owner allowing user data at any desired level of granularity to be resolved based on ownership.

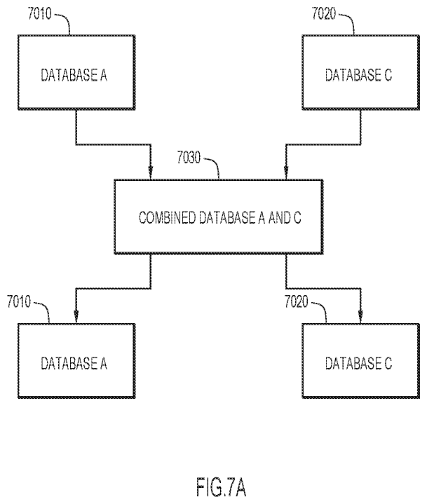

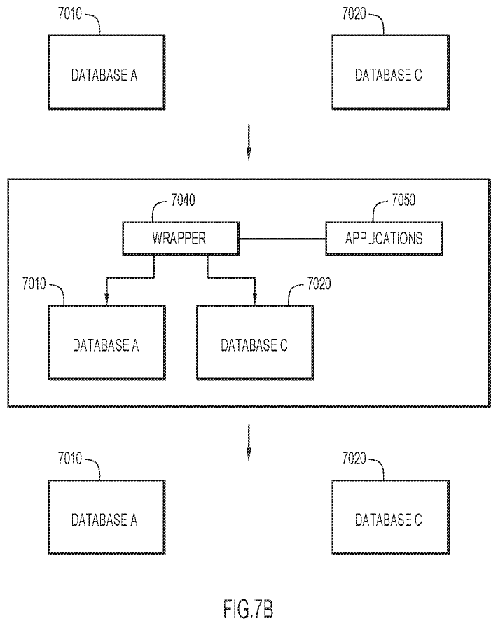

FIGS. 7A and 7B show examples of shared databases. Both examples provide approaches for avoiding data loss. When the shared database is generated, data structures are provided for associating or linking user data with the origin of the user data to allow for subsequent data separation. Both of these examples may be governed by shared data module 64. In terms of providing protecting from data loss, present techniques ensure that user and organization records are only subject to logical (rather than physical) deletes. This protects the association with user data in the event that a user is deleted (for example, due to leaving the organization).

FIG. 7A shows an example implementation in which data from separate databases are imported into a combined database. In this example, the data may be associated with origin information upon import. For example, user data such as notes 2450(a) from organization A may be stored in database A 7010. Notes 2450(c) from organization C may be stored in database C 7020. Both database A and database C may be imported into combined database A and C 7030. In this arrangement, each imported field may be associated with a respective origin tag, field, or metadata for linking the data (e.g., on a per record or per field basis) to its origin. In some aspects, a record in the combined database may contain data pertaining to a user, with a set of notes from organization A and another set of notes from organization C. Upon receiving a revocation, a static copy of the data is provided to both organization A and organization C. In one embodiment, data associated with organization A is exported and stored in an isolated database 7010 in which only organization A may access. Similarly, data associated with organization C is exported and stored in an isolated database 7020 in which only organization C may access. In this embodiment, data is not lost or destroyed during sharing or resolution into separate databases.

FIG. 7B shows another example of a shared database. In this example, the shared database is generated by encapsulating each organizational database in a file wrapper 7040. The file wrapper allows applications 7050 to access data in the underlying databases (e.g., databases 7010 and 7020). In an aspect, the data may reside in each separate organizational database (associated with its respective organization), and the file wrapper may act as an interface between the application accessing data and the underlying database in which the data is housed. The file wrapper 7040 may contain or be associated with one or more parameters for identifying each database, and thus, the origin of the underlying data. Queries provided to the file wrapper return results from both databases. For example, user data such as notes 2450(a) from organization A may reside in database A 7010 owned by organization A. Notes 2450(c) from organization C may reside in database C 7020 owned by organization C. Additional data may be added by respective organizations, and associated with respective databases. The wrapper is able to access underlying data from each respective database, which may utilize different structural languages, thereby integrating data to provide a shared database. Upon receiving a revocation, a static copy of the data is provided to both organization A and organization C and the file wrapper may be removed to disconnect each database. Added data may persist within the database that is associated with the origin of the data. Thus, removal of the file wrapper converts the shared database into isolated databases. In this embodiment, data is not lost or destroyed during resolution.

These examples are not intended to be limited to any particular database.

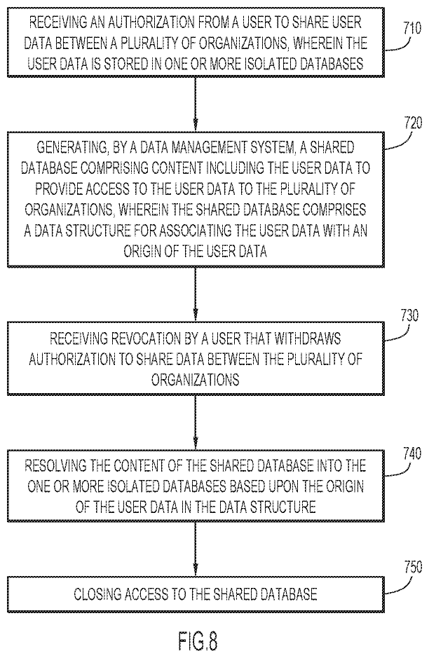

FIG. 8 is a flowchart of operations for managing sharing of data for a user. At operation 710, an authorization from a user to share user data between a plurality of organizations is received, wherein the user data is stored in one or more isolated databases. In an isolated database, the organization that generates the user data and corresponding database has access to the data, but this data is not shared with other organizations.

At operation 720, a medical records data management system generates a shared database comprising content including the user data. The shared database, which may be a logically linked database, provides access to the user data for the plurality of organizations. The shared database comprises a data structure for associating the user data with the respective organization from which the user data is obtained. Thus, for data that originates in the isolated database, the system may utilize the data structure to associate that data with its respective organization (e.g., organization that generates the data, origin of data, etc.). The data structure may capture any information associated with the user data that allows the user data to be associated with the organization that generated the data (e.g., name of organization (e.g., medical practice, hospital, etc.), name of physician, name of database, etc.). This information is used to resolve data upon issuance of a revocation.

At operation 730, a revocation is received by a user that withdraws authorization to share data between the plurality of organizations. Once this occurs, at operation 740, the content of the shared database is resolved into the one or more isolated databases, based upon the origin of the user data in the data structure. Portions of content are assigned to the isolated database based on the data structure. Thus, user data will be returned to the organization that generated the user data (e.g., from the isolated database) or will be returned to the organization that generated the user data within the shared environment. For example, if an organization generates user data within the shared environment, the additional generated data will be associated with the organization that generated such data.

At operation 750, access to the shared database is closed. Once this occurs, organizations are no longer able to access the shared database. In some instances, the shared database is inactivated, while in other instances, the shared database is deleted.

These techniques provide an improvement to the functioning of the computer, in this case, database operations and structure. These techniques provide new data structures for sharing data and unwinding shared data by tracking the origin of information. Database structures may track user data based on origin. Unlike existing approaches, this allows data to be reversibly shared without losing or destroying data. By associating user data with a data structure that captures information regarding origin, the data may be shared and then resolved into components based on origin at any desired level of granularity. Accordingly, data may be combined and then separated in a reversible manner (and later reshared as needed).

These techniques may be applied to a wide variety of environments, including any system in which sharing of information stored in databases is useful.

It will be appreciated that the embodiments described above and illustrated in the drawings represent only a few of the many ways of implementing embodiments for sharing medical records in a manner that is reversible and preserves origin information of the data.

The environment of the present invention embodiments may include any number of computer or other processing systems (e.g., client or end-user systems, server systems, etc.) and databases or other repositories arranged in any desired fashion, wherein the present invention embodiments may be applied to any desired type of computing environment (e.g., cloud computing, client-server, network computing, mainframe, stand-alone systems, etc.). The computer or other processing system employed by the present invention embodiments may be implemented by any number of any personal or other type of computer or processing system (e.g., desktop, laptop, PDA, mobile devices, etc.), and may include any commercially available operating system and any combination of commercially available and custom software (e.g., browser software, communications software, server software, medical records data management system 15, etc.). These systems may include any type of monitors and input devices (e.g., keyboard, mouse, voice recognition, etc.) to enter and/or view information.

It is to be understood that the software (e.g., medical records data management system 15, comprising authorization/revocation management module 62, data import module 63, shared data module 64, data deduplication module 66, data separation module 70, access point module 72, etc.) of the present invention embodiments may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The software of the present invention embodiments (e.g., medical records data management system 15, comprising authorization/revocation management module 62, data import module 63, shared data module 64, data deduplication module 66, data separation module 70, access point module 72, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., user data, data origin, care plan information, authorizations, revocations, demographics, health data, etc.). The database system may be implemented by any number of any conventional or other databases, data stores or storage structures (e.g., files, databases, data structures, data or other repositories, etc.) to store information (e.g., profiles, categories of contribution, user metrics, mapping of information to users and to categories of contribution, etc.). The database system may be included within or coupled to the server and/or client systems. The database systems and/or storage structures may be remote from or local to the computer or other processing systems, and may store any desired data (e.g., user data, data origin, care plan information, authorizations, revocations, demographics, health data, etc.).

The present invention embodiments may employ any number of any type of user interface (e.g., Graphical User Interface (GUI), command-line, prompt, etc.) for obtaining or providing information (e.g., user data, data origin, care plan information, authorizations, revocations, demographics, health data, etc.), wherein the interface may include any information arranged in any fashion. The interface may include any number of any types of input or actuation mechanisms (e.g., buttons, icons, fields, boxes, links, etc.) disposed at any location to enter/display information and initiate desired actions via any suitable input devices (e.g., mouse, keyboard, etc.). The interface screens may include any suitable actuators (e.g., links, tabs, etc.) to navigate between the screens in any fashion.

The output of the medical records data management system 15 may include any information arranged in any fashion, and may be configurable based on rules or other criteria to provide desired information to a user (e.g., user data, data origin, care plan information, authorizations, revocations, demographics, health data, etc.).

The present invention embodiments are not limited to the specific tasks or algorithms described above, but may be utilized for any application in which user data or any other types of confidential data, may be shared on a temporary basis.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for managing sharing of data comprising:
receiving an authorization from a user to share user data between a plurality of organizations, wherein the user data is stored in one or more isolated databases;
generating, by a data management system, a shared database comprising content including the user data to provide access to the user data for the plurality of organizations, wherein the shared database comprises a data structure for associating the user data with an origin of the user data;
receiving revocation by a user that withdraws authorization to share user data between the plurality of organizations;
resolving the content of the shared database into the one or more isolated databases based upon the data structure; and
closing access to the shared database.

2. The method of claim 1, wherein the shared database is a logically linked database.

3. The method of claim 1, wherein receiving the revocation further comprises:
providing a static copy of the content to each organization of the plurality of organizations.

4. The method of claim 1, wherein the plurality of organizations collaborate on a plurality of treatments and a plurality of care options associated with the user, and wherein the user data comprises demographic data and health data associated with the user.

5. The method of claim 1, wherein closing access to the shared database further comprises:
restricting access of each organization of the plurality of organizations to the content in the shared database.

6. The method of claim 1, wherein resolving the content further comprises:
separating the content into a plurality of isolated databases based on the origin of the user data; and
providing each isolated database to the organization associated with the origin of the user data.

7. The method of claim 1, wherein the user data comprises one or more records associated with a patient, including demographic data, health data, and care plan data for the patient, and wherein authorization includes a plurality of permissions authorizing sharing of specific subsets of user data.

8. The method of claim 1, wherein receiving the revocation further comprises:
generating a plurality of isolated databases, wherein each isolated database generated from the shared database is visible to the organization associated with the origin of user data, and comprises demographics, health data and care plans.

9. The method of claim 1, wherein receiving the authorization further comprises:
copying the user data from each isolated database and storing the copied data locally; and
logically linking the copied data to form a shared database.

10. A computer system to manage sharing of data, the computer system comprising:
one or more computer processors;
one or more computer readable storage media;
program instructions stored on the one or more computer readable storage media for execution by at least one of the one or more computer processors, the program instructions comprising instructions to:
receive an authorization from a user to share user data between a plurality of organizations, wherein the user data is stored in one or more isolated databases;
generate a shared database comprising content including the user data to provide access to the user data for the plurality of organizations, wherein the shared database comprises a data structure for associating the user data with an origin of the user data;
receive revocation by a user that withdraws authorization to share user data between the plurality of organizations;
resolve the content of the shared database into the one or more isolated databases based upon the data structure; and
close access to the shared database.

11. The system of claim 10, wherein the program instructions further comprise instructions to:
provide a static copy of the content to each organization of the plurality of organizations.

12. The system of claim 10, wherein the program instructions further comprise instructions to:
restrict, when the shared database is closed, access of each organization of the plurality of organizations to the content in the shared database.

13. The system of claim 10, wherein the program instructions, in order to resolve the content, further comprise instructions to:
separate the content into a plurality of isolated databases based on the origin of the user data; and
provide each isolated database to the organization associated with the origin of the user data.

14. The system of claim 10, wherein the program instructions further comprise instructions to:
generate, upon receiving the revocation, a plurality of isolated databases, wherein each isolated database generated from the shared database is visible to the organization associated with the origin of user data, and comprises demographics, health data and care plans.

15. The system of claim 10, wherein the program instructions further comprise instructions to:
copy, upon receiving the authorization, the user data from each isolated database and store the copied data locally; and
logically link the copied data to form a shared database.

16. A computer program product to manage sharing of data, the computer program product comprising one or more computer readable storage media collectively having program instructions embodied therewith, the program instructions executable by a computer to cause the computer to:
receive an authorization from a user to share user data between a plurality of organizations, wherein the user data is stored in one or more isolated databases;
generate, by a data management system, a shared database comprising content including the user data to provide access to the user data for the plurality of organizations, wherein the shared database comprises a data structure for associating the user data with an origin of the user data;
receive revocation by a user that withdraws authorization to share user data between the plurality of organizations;
resolve the content of the shared database into the one or more isolated databases based upon the data structure; and
close access to the shared database.

17. The computer program product of claim 16, wherein the program instructions further comprise instructions to:
provide a static copy of the content to each organization of the plurality of organizations, and
restrict, when the shared database is closed, access of each organization of the plurality of organizations to the content in the shared database.

18. The computer program product of claim 16, wherein the program instructions, in order to resolve the content, further comprise instructions to:
separate the content into a plurality of isolated databases based on the origin of the user data; and
provide each isolated database to the organization associated with the origin of the user data.

19. The computer program product of claim 16, wherein the program instructions further comprise instructions to:
generate, upon receiving the revocation, a plurality of isolated databases, wherein each isolated database generated from the shared database is visible to the organization associated with the origin of data, and comprises demographics, health data and care plans.

20. The computer program product of claim 16, wherein the program instructions, further comprise instructions to:
copy, upon receiving the authorization, the user data from each isolated database and store the copied data locally; and
logically link the copied data to form a shared database.
